(19) United States
(12) Patent Application Publication
(10) Pub. No.: US 2011/0053271 A1
(43) Pub. Date: Mar. 3, 2011

(54) IRRADIATION INDUCED GRAFTING OF POLYSACCHARIDES TO CELL CULTURE VESSELS

(76) Inventors: Odessa N. Petzold, Elmira, NY (US); Vasudha Ravichandran, Painted Post, NY (US); Wageesha Senaratne, Horseheads, NY (US)

(21) Appl. No.: 12/547,144

(22) Filed: Aug. 25, 2009

(51) Int. Cl. C12N 5/06; B05D 3/06

(52) U.S. Cl. 435/404; 427/533; 427/535

(57) ABSTRACT

A method for grafting a polysaccharide to a surface of a cell culture article includes (a) contacting the surface of the article with the polysaccharide in a dry form; and (b) exposing the surface of the article and contacted dry polysaccharide to ionizing radiation to graft the polysaccharide to the surface of the article. In addition to grafting the polysaccharide to the surface, the ionizing radiation may serve to sterilize the article.

### FIELD

The present disclosure relates to cell culture articles and methods; and more particularly to methods and articles having galactomannan cell culture substrates.

### BACKGROUND

Specialized cell culture surfaces that support cells having more in vivo-like characteristics in cell culture are desirable for many applications. In some instances, these cell culture surfaces are more useful when they don't degrade or delaminate in standard cell culture conditions. In addition, these cell culture surfaces must be sterilized or sterilizable.

### BRIEF SUMMARY

The present disclosure describes, among other things, the use of ionizing radiation to graft solid state polysaccharides to cell culture substrates, immobilizing the polysaccharides to the cell culture article, and improving the integrity of the polysaccharide coating under cell culture conditions. In various embodiments, a galactomannan is grafted to the surface of a cell culture article, and a small percentage of the galactomannan that does not bond to the substrate during radiation is broken down into low molecular weight extractables believed to be oligomeric sugars and galactose or mannose, or derivatives thereof that are not harmful to cultured cells. These extractables may be present in the composition of some cell culture media, and thus do not, in many embodiments, have to be removed prior to culturing cells. In addition, to reduce post-irradiation chemistry steps to achieve a robust cell culture surface, grafting by irradiation may also serve to sterilize the cell culture article, further reducing the number of processing steps to produce a viable cell culture article.

In various embodiments, the present disclosure provides a method for grafting a polysaccharide to a surface of a cell culture article. The method includes contacting the surface of the article with the polysaccharide in a dry form and exposing the surface of the article and contacted dry polysaccharide to ionizing radiation to graft the polysaccharide to the surface of the article. In some embodiments, the polysaccharide may be contacted to the surface in a wet form and then dried prior to exposure to gamma radiation.

In numerous embodiments, the disclosure provides cell culture articles having a substrate surface and a polysaccharide covalently bonded to the surface. The cell culture articles may be used to culture a variety of cells, including liver cells, such as human and non-human primary hepatocytes and hepatocyte cell lines.

Advantages of one or more of the various embodiments presented herein over prior cell culture articles and methods will be readily apparent to those of skill in the art. For example, it will be understood that the dual use of ionizing radiation to graft and sterilize in a single step will, in many circumstances, be considered to be advantageous. Further, the ability to forgo subsequent processing to remove agents, such as cross-linking agents, initiators, and the like, may be considered advantageous. These and other advantages will be readily understood from the following detailed descriptions when read in conjunction with the accompanying drawings.

### BRIEF DESCRIPTION OF THE DRAWINGS

FIGS. 1A-B are schematic diagrams of side views of portions of cell culture articles having polysaccharide cell culture substrate layers.

FIGS. 2A-C are schematic diagrams of cross sections of a well of a cell culture article. In FIG. 2A, the well is uncoated. In FIGS. 2B-C, a polysaccharide layer is disposed on at least a portion of the surface of the well.

FIG. 3 is a schematic drawing illustrating a process for grafting a polysaccharide to a surface.

FIGS. 4A-B are flow diagrams illustrating processes for grafting a polysaccharide to a surface.

FIG. 5 is a schematic drawing illustrating a process for grafting a polysaccharide to a surface.

FIGS. 6A-B are flow diagrams illustrating processes for grafting a polysaccharide to a surface.

FIG. 7A is a graph of results of a Gel Permeation Chromatography (GPC) experiment where locust bean gum (LBG) coated cell culture articles were subjected to sterilizing UV radiation, showing leaching of LBG.

FIG. 7B is a bar graph showing the percent of peak area of the GPC experiment for the three UV-treated LBG coated samples at the 11.3 minute peak.

FIG. 8A is a graph of results of a GPC experiment where LBG coated cell culture articles were subjected to gamma radiation (25-40 kGy), showing no leaching of LBG.

FIG. 8B is bar graph showing the percent of peak area of a GPC experiment for three gamma irradiated LBG samples at 13.5 minutes.

FIGS. 9A-B are 50× fluorescent (A) and bright field (B) images of cells cultured on gamma treated LGB surfaces at day 14.

FIG. 10 is a bar graph showing results of GPC experiments LBG coated articles subjected to low (↓) (10-18 kGy) and high (↑) (25-40 kGy) dose gamma radiation LBG coated articles at a retention time of 13.5 minutes.

FIG. 11 is a representative GPC sample of a pH changed (pH 7.4) LBG coated article subjected to high dose gamma radiation at day 1 (D1) and day 10 (D10).

FIG. 12 is a representative GPC sample of a LBG coated article subjected to high dose gamma radiation at day 1 (D1) and day 10 (D10).

FIG. 13 is a graph of GPC results comparing LBG, untreated (u), and LBG film (f), powder (p), and solution (s) subjected to gamma radiation.

FIG. 14 is a graph of GPC results comparing untreated LBG, (u) LBG subjected to gamma radiation (F), and LBG subjected to heat and gamma radiation (F-H).

FIG. 15 is an optical image of a polystyrene 96 well plate coated with LBG and pre- and post-treated with electron beam radiation. The image was taken after four days of soaking in buffer and crystal violet staining.

FIG. 16 is a bar graph showing the weight of locust bean gum dissolved in PBS after a 3 days of soaking at 37° C. after various pretreatment and post-treatment conditions of e-beam irradiation.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.” It will be understood that “consisting essentially of”, “consisting of”, and the like are subsumed in “comprising” and the like. Accordingly, a galactomannan layer comprising a galactomannan polymer may be a galactomannan layer consisting essentially of, or consisting of, a galactomannan polymer.

As used herein “galactomannan polymer” means a polysaccharide having a mannose backbone with galactose side groups. Galactomannan polymers may form a part of, or all of, a galactomannan coating, galactomannan substrate, or galactomannan layer as described herein.

The present disclosure describes, inter alia, the use of ionizing radiation to graft polysaccharides, such as galactomannan polymers, to cell culture substrates, immobilizing the polysaccharides to the cell culture article, and improving the integrity of the polysaccharide coating under cell culture conditions. Multiple post chemistry steps, such as washing, extraction or subsequent sterilization, associated with other grafting processes, such as those employing chemical crosslinkers or polymerization initiators, may be eliminated to achieve a robust cell culture surface and provide sterilization in one step.

Galactomannan coated cell culture vessels have been shown to provide suitable surfaces for liver cell growth and function. However, these coatings undergo significant swelling (8-10 times) under cell culture conditions that can result in random delamination of the coating from the vessel substrate. In part this may be due to limited interface between the coating and the substrate, which is believed to be driven by secondary forces of interaction (Van der Waals, etc.) and physical interactions (roughness, etc). Significant swelling also leads to compromised integrity of the coating via slow dissolution or leaching of the galactomannan polymers into the cell culture medium, resulting in gradual cell loss or diminished cell retention over time.

Polysaccharides, including galactomannans, usually degrade when exposed to ionizing radiation. For example, the galactomannan, guar gum, degrades in solution state when subjected to low gamma radiation with the rate of chain breaks being higher at lower irradiation doses than those at higher doses. In solid state, the galactomannans, guar gum, tara gum and locust bean gum were observed to depolymerize when exposed to a ⁶⁰Co-gamma cell, showing a pseudoplastic behavior until a certain dose and then showing Newtonian flow while loosing the gelling properties.

Ionizing radiation has been used as a tool for modification of polymer materials through cross-linking. For example, gamma irradiation has been shown to increase the mechanical properties of collagen films in the presence of carbohydrates, including cellulose, chitin and galactomannan, by including cross-links between the protein chains. By way of further example, high concentration aqueous solutions of hydrogels of carboxymethyl cellulose, carboxymethyl chitin, carboxymethyl chitosan or carboxymethyl starch, in so-called “paste-like” condition of a polymer, have been shown to undergo crosslinking when exposed to gamma radiation. In another example, hydrocolloids (e.g. galactomannans such as locust bean gum) have been utilized simultaneously with starch to form films in order to modify material mechanical resistance. In these examples, gamma irradiation was used either (1) to generate a composite film of a carbohydrate (e.g. starch, cellulose, galactomannan) and a protein, peptide or synthetic polymer with improve mechanical properties, or (2) to graft the carbohydrate (e.g. starch, cellulose, galactomannan) to other polymers in solution. To date, there has been no disclosure describing immobilizing (grafting) galactomannan film or coating to a solid support through the use of gamma radiation, particularly in a dried solid state.

Electron beam irradiation has been used for reagent-free crosslinking of polysaccharides, primarily for application in wound dressings. For example, electron beam irradiation has been used to form hydrogels from carboxymethyl starch and to crosslink polyvinyl pyrrolidone and carboxymethyl chitosan to enhance the antibacterial activity and protein absorptive property of hydrogels. Electron beam irradiation has also been shown to enhance the biodegradability of crosslinked hydroxypropylmethyl cellulose, and has been shown to result in crosslinking of Arabic gum at polysaccharide concentrations as low as 10% and produced gels safe for food applications. However, these examples describe the use of e-beam radiation for crosslinking of carbohydrates and other polymers in solution only.

In the cell culture vessel industry, ionizing radiation is typically used for sterilization purposes only. However, as described herein, ionizing irradiation may be employed not only to provide sterilization of the vessel, but also to enable grafting of polysaccharides to a cell culture vessel substrate capable of producing free radicals and susceptible to free radical addition reaction when exposed to ionizing radiation, such as gamma radiation or e-beam radiation. Use of such radiation for grafting as described herein may reduce delamination of the polysaccharide from the cell culture substrate or reduce leaching of polysaccharide under cell culture conditions.

Any cell culture vessel having a surface capable of producing free radicals and susceptible to free radical addition reaction when exposed to ionizing radiation may be employed in accordance with the teachings herein. For example, plastics or polymers, including dendritic, branched, linear and comb polymers. Some examples are polystyrenes and derivatives of poly(styrene), poly(vinyl chloride), poly(vinyl alcohol), poly(vinyl acetate-maleic anhydride), poly(dimethylsiloxane), cyclic or acyclic olefin polymers, fluorocarbon polymers, polypropylene, polyethyleneimine, poly(vinylpyrrolidone), poly(meth)acrylamide, poly(meth)acrylate, copolymers such as poly(vinyl acetate-co-maleic anhydride), poly(styrene-co-maleic anhydride), poly(ethylene-co-acrylic acid) or derivatives of these or the like, may be able to produce free radicals and may be susceptible to free radical addition when exposed to ionizing radiation.

In some embodiments, an intermediate coating having a surface capable of producing free radicals and undergoing free radical addition is disposed between the base material and the polysaccharide to allow grafting of the polysaccharide. Such an intermediate coating may be desirable when the base material is not capable of producing sufficient free radicals and undergoing sufficient free radical addition. For example, it may be desirable to employ a suitable intermediate layer when the base material of the cell culture vessel is silicon or a glass material such as soda-lime glass, pyrex glass, vycor glass, or quartz glass. Preferably, the intermediate material can sufficiently bond to the base material so that it does not delaminate under cell culture conditions. By way of example, epoxy coatings or alkyl and allyl silanes or silsesquioxanes may be applied to glass or other surfaces, or polyamide, polyimide, polystyrene, polypropylene, or polyethylene layers or the like may be applied to polymer base materials or other materials, as intermediate coating layers.

In various embodiments, the surface of a base material or intermediate coating is treated, either physically or chemically, to enhance the ability of the surface to graft to the polysaccharide. For example, the surface may be corona treated or plasma treated.

Examples of vacuum or atmospheric pressure plasma include RF and microwave plasmas both primary and secondary, dielectric barrier discharge, and corona discharge generated in molecular or mixed gases including air, oxygen, nitrogen, argon, carbon dioxide, nitrous oxide, or water vapor.

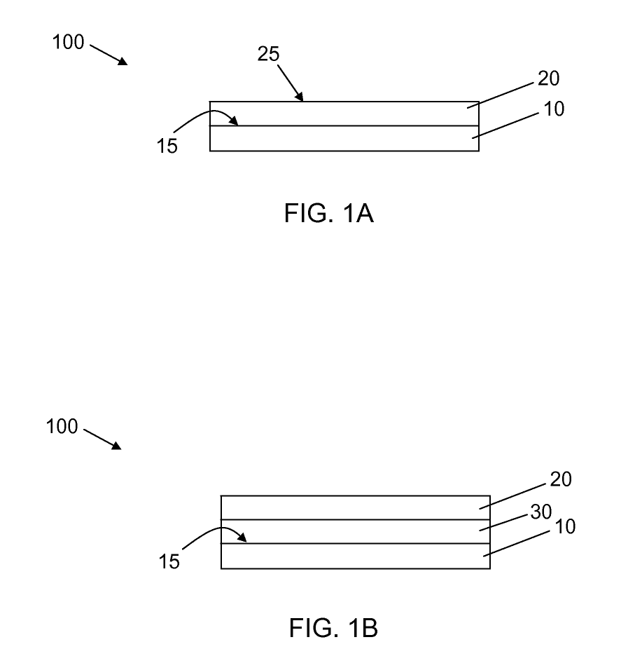

Referring now to FIGS. 1A-B, schematic diagrams of articles 100 for culturing cells are shown. The article 100 includes a base material substrate 10 having a surface 15. A layer of polysaccharide 20 is disposed on the surface 15 of the base material 10. The polysaccharide layer 20 has a top surface 25. While not shown, it will be understood that synthetic polymer coating 20 may be disposed on a portion of base material 10. As shown in FIG. 1B, an intermediate layer 30 may be disposed between surface 15 of base material 10 and the layer of polysaccharide 20. While not shown, it will be understood that polysaccharide 20 may be disposed on a portion of intermediate layer 30. It will be further understood that intermediate layer 30 may be disposed on a portion of base material 10.

Non-limiting examples of articles 100 suitable for cell culture include single and multi-well plates, such as 6, 12, 96, 384, and 1536 well plates, jars, petri dishes, flasks, multi layer flasks (HYPERFlask®), CellSTACK®, Cellcube®, beakers, plates, roller bottles, slides, such as chambered and multichambered culture slides, tubes, cover slips, cups, spinner bottles, perfusion chambers, bioreactors and microcarriers such as beads and fermenters.

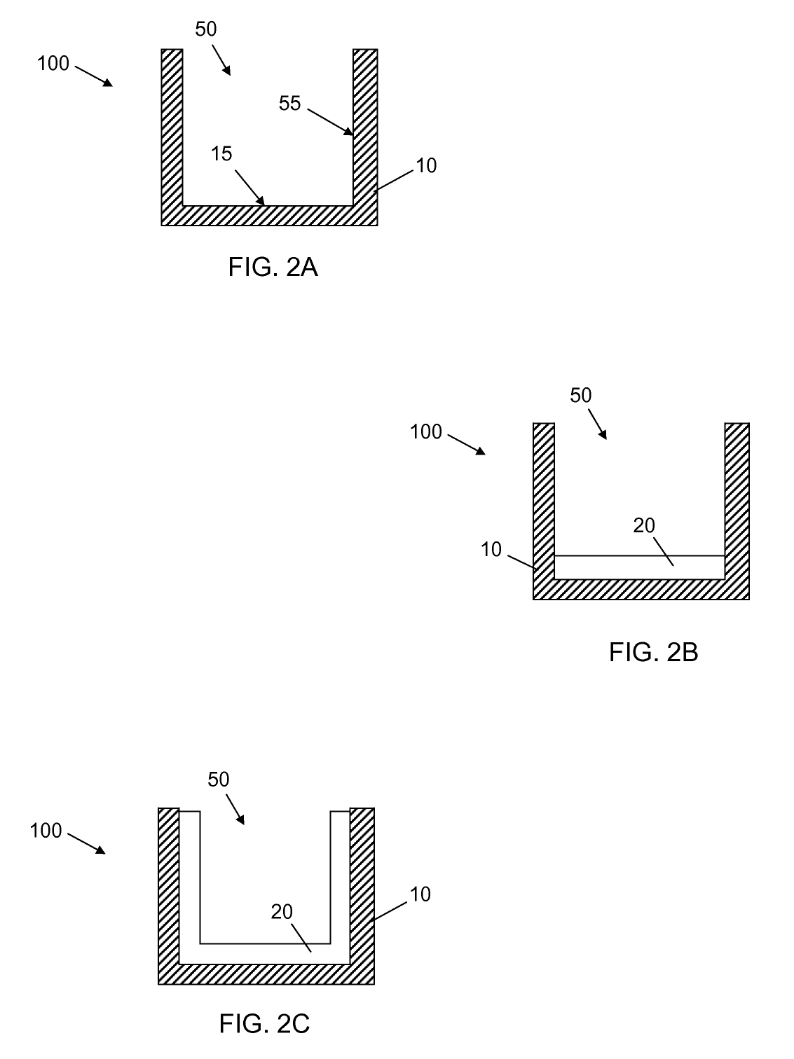

Referring now to FIG. 2A-C, article 100, in numerous embodiments, is cell culture ware having a well 50, such as a petri dish, a jar, a bottle, a multi-well plate, a flask, a beaker, a fermenter, a bioreactor or other vessel having a well. The article 100 is formed from base material 10 may include one or more wells 50. Well 50 includes a sidewall 55 and a surface 15. Referring to FIG. 2B-C, a polysaccharide 20 may be disposed on surface 15 or sidewalls 55 or a portion thereof, or, as discussed above with regard to FIG. 1 one or more intermediate layer 30 may be disposed between surface 15 or sidewall 55 and polysaccharide 20.

Polysaccharide layer 20, whether disposed on an intermediate layer 30 or base material 10, in various embodiments, uniformly coats the underlying substrate. By “uniformly coated,” it is meant that the layer 20 in a given area, for example a surface of a well of a culture plate, completely coats the area at a thickness of about 5 nm or greater. While the thickness of a uniformly coated surface may vary across the surface, there are no areas of the uniformly coated surfaces through which the underlying layer (either intermediate layer 30 or base material 10) is exposed. Of course, the polysaccharide layer 20 may be non-uniformly coated.

Polysaccharide layer 20 may have any desirable thickness. Polysaccharide that is not grafted to the surface of the cell culture article may tend to delaminate. See US Patent Application Publication No 2008/0220526 which discloses polysaccharide layer at thicknesses up to 1 mm. However, grafting thicknesses can be 1 mm or greater than 1 mm. For example, the polysaccharide layer may have a thickness of greater than 2 mm, greater than about 2.5 mm, or greater than about 3 mm. Of course, the polysaccharide may have a thickness of less than 1 mm, such as less than 0.5 mm, less than 0.25 mm, less than 0.1 mm, or less than 0.05 mm. Thickness may be an average thickness of measurements taken from multiple sites.

Polysaccharide Layer

Any suitable polysaccharide, or combinations of polysaccharides, may be grafted to a cell culture surface using ionizing radiation as described herein. In various embodiments, the polysaccharide layer includes a galactose based polysaccharide (i.e., a polysaccharide having repeating units of galactose, or galactose included in branch points or a derivative thereof). A galactose-based polysaccharide, or other polysaccharide, may be a homopolysaccharide (where all of the monosaccharides of the polysaccharide are of the same type of monosaccharide) or may be a heteropolysaccharide (where the polysaccharide is formed from more than one type of monosaccharide). Examples of naturally occurring galactose based polysaccharides include locust bean gum (also known as carob bean gum, carob seed gum, carob gum), guar gum, cassia gum, tragacanth gum, tara gum, karaya gum, gum acacia (gum Arabic), ghatti gum, cherry gum, apricot gum, tamarind gum, mesquite gum, larch gum, psyllium, fenugreek gum, xanthan gum, seaweed gum, gellan gum, agar gum, cashew gum, carrageenan and curdlan. Of course, any other suitable polysaccharide or combination of polysaccharides for which it may be desirable to form a surface on which to culture cells may be employed in accordance with the teachings presented herein.

In various embodiments, a galactomannan polymer is grafted to a cell culture surface using ionizing radiation. For example, the galactomannan polymer may have a mannopyranose backbone with branch points from their 6-positions linked to galactose residues, such as α-D-galactose. A representative segment of a galactomannan polymer showing a portion of the mannose backbone and a galactose side group is shown in the following:

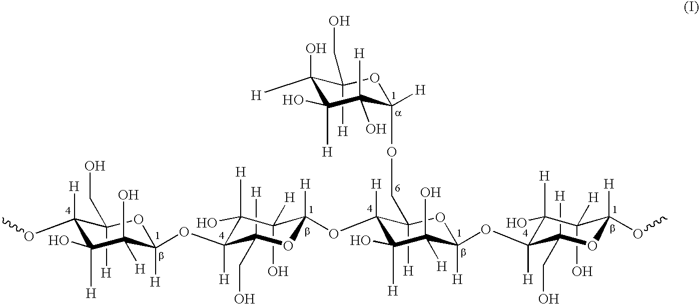

Suitable galactomannan polymers may have any suitable ratio of mannose to galactose. For example, the mannose-to-galactose ratio may be from about 1:1 to about 6:1. Suitable galactomannan polymers include include locust bean gum, (also known as carob bean gum, carob seed gum, carob gum), guar gum, cassia gum, tara gum, mesquite gum, fenugreek gum, and their derivatives. Locust bean gum (LBG) has about 4 mannose residues for every galactose residue (a mannose-to-galactose ratio of about 4:1). Guar gum has a mannose/galactose ratio of about 2. The mannose-to-galactose ratio is about 1:1 for mesquite gum and fenugreek gum, about 3:1 for tara gum and about 5:1 for Cassia gum. Guar gum, like LBG, is a galactomannan polysaccharide consisting of a mannopyranose backbone. However, guar gum has more galactose branchpoints than LBG. This difference in structure causes guar gum to be more soluble and to have a higher viscosity than LBG. That is, LBG is a galactomannan polymer with lower galactose substitution and therefore it is “less stiff”. The larger the mannose-to-galactose ratio, the less viscous and less soluble the gum. Therefore, gums with a higher mannose-to-galactose ratio are less stiff and, more flexible, while gums with a lower mannose-to-galactose ratio are better subject to solubility, dispersion and emulsification. LBG has more flexibility (lower modulus) than guar gum. Higher galactose substitution gives these gums improved solubility, dispersiveness and emulsification. Higher galactose substitution makes the galactomannan polysaccharides stiffer. Higher substitution lends the gum characteristics of higher viscosity and higher solubility.

Galactomannan gums are galactomannan polysaccharides that may be derived from natural sources, from recombinants or synthetic sources, or form combinations of natural, recombinant or synthetic sources. Galactomannan gums include gums that are purified or treated or modified by processes which may include enzymatic treatment, filtration, centrifugation, hydrolysis, freeze-thaw cycles, heating and chemical treatments or modifications, mixtures of galactomannan gums with other galactomannan gums or non-galactomannan gums, or with other ingredients that enhance the cell culture characteristics of the galactomannan layer.

Some naturally occurring gums can be produced as exudates of plants which may be produced by plants after a plant has been wounded. A wounded gum-producing plant produces an exudate in response to a wound (tears of gum). These kinds of plant exudates may be harvested by wounding a plant or a plant seed and removing the exudates. These exudates can then be cleaned to remove dust, dirt and other impurities. The exudates may be dried, powdered and suspended in liquid. Further processing steps may include enzymatic treatment, filtration, centrifugation, hydrolysis and other chemical modifications.

Galactomannan gums can also be derived from plant seeds. Seed-derived gums include guar (Cyamopsis tetragonoloba), locust bean or carob bean (Ceratonia siliqua), tara (Caesalpinia spinosa), fenugreek (Trigonella foenum-graecom), mesquite gum (Prosopis spp) and Cassia gum (Cassia tora). Gums are generally derived from the endosperm of the seeds. After removal of the shell and the germ of the seed, the endosperm is ground. The resulting powder (flour) can contain a high polysaccharide content which, upon mixing with water and/or other ingredients, can form a liquid gum substance. The suspension formed upon mixing the powdered gum with liquid can contain impurities such as cellulose, other plant matter, and various chemical impurities. The suspension can be treated to increase the purity of the gum suspension. For example, the liquid can be centrifuged, filtered, heated, or put through freeze-thaw cycles to remove impurities.

Powdered gum raw materials may also be purified or filtered according to particle size. For example sieves can be used to separate powdered gum material according to particle size. Particle sieve size openings of 250, 106, 53, 38 and 25 μm can be used to separate powdered gum material to create filtered particle size mixtures of between 106 and 250 μm, between 53 and 106 μm, between 38 and 53 μm, and between 25 and 38 μm particles. These separated particle sizes, or mixtures of these particle sizes, can be used to produce glactomannan coatings.

Gum material can be purchased from chemical supply houses including Sigma-Aldrich, Fluka, TIC-Gums Inc. (Belcamp, Md.), Gum Technology Corporation and Herchles (formerly Aqualon) Wilmington, Del. Because these gums are used as food additives, and for industrial applications, these materials may be provided in less pure forms than might be desired for some uses. Therefore, additional purification steps and additional treatments may be desired. Purification steps may include, for example, extraction in a solvent that is less polar than water, for example, ethanol.

Galactomannan gums may be derived from recombinant or synthetic sources. For example, galactomannose may be synthesized in vivo from GDP-mannose and UDP-galactose by the enzymes mannan synthase and galactosyltransferase. DNA coding for these proteins has been isolated and characterized, (US Patent Publication 2004/0143871) and recombinant plants transformed with these enzymes have been shown to express elevated levels of galactomannan. In addition, the degree of galactosylation of the mannopyranose backbone may be influenced by the presence (or absence) of alpha-galactosidase in vivo, (see Edwards et al. Plant Physiology (2004) 134: 1153-1162). Alpha-galactosidase removes galactose residues from the mannopyranose backbone. For example, seeds that naturally express galactomannans with a lower degree of galactosylation may express (or express more) alpha galactosidase, which removes galactose moieties from the mannopyranose backbone in those species of plant. The alpha-galactosidase enzyme may be used to reduce the presence of galactose on the mannopyranose backbone of naturally occurring galactomannose gums in a laboratory manipulation of the characteristics of the naturally occurring galactomannose gum. Accordingly, gums and galactomannose gums, whether naturally occurring, recombinant, or synthetically produced, may be treated with alpha-galactosidase to reduce the presence of galactose on the mannopyranose backbone. Glactomannan gums that can be useful as coatings grafted to cell culture articles include those that have been treated with alpha-galactosidase or other enzymes or chemical treatments, to “tune” the gums to provide the gum with desired characteristics as a coating for cell culture surfaces.

Polysaccharide layers may include more than one gum material. The combined gum material can include, for example, one or more of locust bean gum (also known as carob bean gum, carob seed gum, carob gum), guar gum, cassia gum, tragacanth gum, tara gum, karaya gum, gum acacia (gum Arabic), ghatti gum, cherry gum, apricot gum, tamarind gum, mesquite gum, larch gum, psyllium, fenugreek gum, xanthan gum, seaweed gum, gellan gum, agar gum, cashew gum, carrageenan and curdlan.

Polysaccharide layers may include galactomannans or other gums that have been modified to optimize physical and chemical characteristics of the cell culture coatings made from the gum compounds. Physical and chemical characteristics of the cell culture coatings may be “tuned” by chemically modifying the gum material. “Tuned” or “tunable” used here means that gum material, obtained naturally or synthetically, can be modified, either physically or chemically to adjust the physical or chemical characteristics of the material to provide a cell culture substrate that promotes a desired cell culture environment. In various embodiments, the galactomannans or other gums of the polysaccharide layer are tuned to provide a preferred cell culture environment for a particular cell type growing in a particular type of cell culture media, for a particular purpose.

Physical characteristics of the material can be changed by changing the porosity of the coating material, changing the modulus of the coating, changing the charge density and distribution, surface energy, topology and porosity, or by changing other physical characteristics of the matrix. Chemical characteristics of the material can be changed by providing chemical crosslinkers in the material, adding or removing chemical groups from a particular matrix material, mixing different gum substances together, changing the type and density of receptor ligands present in the cell culture environment or by other chemical modifications. Chemical modifications can change the physical characteristics of a gum coating material, and physical modifications can involve chemical modifications. In various embodiments, the cell culture surface coating is tuned by, for example: changing the modulus of the naturally-occurring or synthetic material, changing the freeze-thaw conditions used to provide the matrix, changing the porosity of the material, crosslinking the polysaccharide compounds, mixing multiple gum compounds together, adding additional compounds to the gum material in the coating material, changing the number or nature of the sidechains present on the backbone of polysaccharide gum material in the coating, substituting sidechains, treating with enzymes to change the chemical nature of the material, or by any other method.

In some embodiments, the number and nature of galactose sidechains in a polysaccharide cell culture surface coating may be changed. This modification affects the properties of galactose-presenting polysaccharide-based matrices. For example, polysaccharide cell culture surface coatings including galactomannan polysaccharide coatings are tunable by adding and removing galactose polysaccharide sidechains from galactomannan polysaccharide gum cell culture surface coatings, and by changing the number of mannose groups per galactose in a galactomannose polysaccharide, or by changing the nature of these sidechains.

In various embodiments, the cell culture surface coating is treated or tuned to make a physically crosslinked system. A crosslinked gum is a physical gel which can be a gel, a hydrogel, a film or a hydrofilm. A hydrofilm is a thin, transparent or nearly transparent coating. Physical and chemical treatments may cause a cell culture surface coating to become cross-linked. Physical treatments include exposure to particular temperature ranges, including heat, cold and freeze-thaw heat and cold cycles. Solutions of locust bean gum will gel under cryogenic treatment (freezing-thawing cycles). These freeze-thaw cycles create physical-gels or physically crosslinked networks. A solution of locust bean gum, if kept at room temperature will remain fluid. After prolonged storage (2-3 months) the solution will form a weak physical gel.

Polysaccharide coatings may be tuned to vary the modulus of the polymer coating. Embodiments of tuned gums of the present invention are gums which have been centrifuged, filtered, heated, frozen, freeze-thawed, chemically or enzymatically treated, exposed to light or otherwise altered, to improve the cell culture characteristics of a cell culture substrate coating made from the treated gum. Tuned gums also include gums which are in gel, hydrogel, film or hydrofilm form. Further, tuned gums include galactomannan gums which have been centrifuged, filtered, heated, frozen, freeze-thawed, enzymatically treated, or otherwise altered, and which are in gel, hydrogel, film or hydrofilm form for use as a coating for cell culture. In some embodiments, the coatings are not smooth, but include “bumps” or “pellets” of gum material on a cell culture surface.

The polysaccharide layer may include mixtures of gums. Mixtures of gums can form cross-linked gel compositions. For example, charged polysaccharides such as xanthan gum or carrageenan or neutral polysaccharides with gelling behavior, such as agar or curdlan, can be mixed with galactomannan gums to form a blend that forms a gel at room temperature.

In some embodiments, the polysaccharide layer materials have been tuned to change the charge characteristics of the coating, which may be accomplished by changing the charge blending linear or branched charged polysaccharides in the coating. Charged polysaccharides such as xanthan gum or carrageenan are mixed with a naturally occurring gum(s) to form a blend. Combinations of galactomannan gums with other gums such as carageenan and xanthan gums (charged polysaccharides) are capable of synergistic interactions. These gums in combination with locust bean gum form thermoreversible soft elastic gels without any cryogenic (freeze-thaw) treatment. A greater proportion of guar gum (80:20) may result in enhanced synergy for room temperature gellation compared to locust bean gum (50:50). In embodiments, mixtures of guar gum; charged polysaccharide ranging from 70:30 to 90:10, and mixtures of locust bean gum; charged polysaccharides ranging from 40:60 to 60:40 are provided.

In some embodiments, a mixture (or blend) of at least two types of gum polysaccharides can be used to coat the surface of a substrate to form a cell-culture-friendly coating. Coating a substrate with these gum polysaccharides individually or in a mixture of any combination of these polysaccharides can result in a matrix presenting varied amounts of galactose or other polysaccharide moieties. Mixtures or blends of at least two gums may include, for example, mixtures of any two or more gums from the following: guar gum, locust bean gum (also known as carob bean bum, carob seed gum and carob gum), cassia gum, tragacanth gum, tara gum, karaya gum, gum acacia (also known as gum arabic), ghatti gum, cherry gum, apricot gum, tamarind gum, mesquite gum, larch gum, psyllium, fenugreek gum and tara gum. Gums that are derived from bacterial and algal sources include xanthan gum, seaweed gum, gellan gum, agar gum, carrageenan and curdlan.

Other properties of a polysaccharide gum matrix may be further or alternatively tuned. By way of further example, charged polysaccharides such as xanthan gum or carrageenan may be mixed with a naturally occurring gum(s) to form a blend. The resultant blend is used to form a matrix having controlled charge density for cell culturing, including hepatocyte culturing. In addition or alternatively, linear neutral polysaccharides with gelling behavior, such as agar or curdlan, may be mixed with the naturally occurring branched polysaccharide(s) to form a blend. The resultant blend may be used to form a matrix having material properties such as modulus and stability for long term cell culturing.

Polysaccharide coating layers may include one or more biologically active molecule. Inclusion of a biologically active molecule may promote cell adhesion, proliferation or survival. Or, the inclusion of a biologically active molecule might improve function of cells in culture. Bioactive molecules include human or veterinary therapeutics, nutraceuticals, vitamins, salts, electrolytes, amino acids, peptides, polypeptides, proteins, carbohydrates, lipids, polysaccharides, nucleic acids, nucleotides, polynucelotides, glycoproteins, lipoproteins, glycolipids, glycosaminoglycans, proteoglycans, growth factors, differentiation factors, hormones, neurotransmitters, pheromones, chalones, prostaglandins, immunoglobulins, monokines and other cytokines, humectants, minerals, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules. Glycoaminoglycans include glycoproteins, proteoglycans, and hyaluronan. Polysaccharides include cellulose, starch, alginic acid, chytosan, or hyaluronan. Cytokines include, but are not limited to, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, interleukin (IL) 1, IL-2, IL-3, IL-4, IL-5, IL-6, IL-7, IL-8, IL-9, IL-10, I-11, IL-12, IL-13, TNF-alpha, and TNF-beta. Immunoglobulins useful herein include, but are not limited to, IgG, IgA, IgM, IgD, IgE, and mixtures thereof Amino acids, peptides, polypeptides, and proteins can include any type of such molecules of any size and complexity as well as combinations of such molecules.

Examples include, but are not limited to, structural proteins, enzymes, and peptide hormones. These compounds can be mixed with the gum compounds as they are being prepared, or can be added to the surface of the cell culture coating after it has been applied to a cell culture surface. For a more detailed discussion of suitable biologically active molecules that may be employed, see U.S. Patent Application Publication No. 2008/0220526, published on Sep. 11, 2008, naming Ellison et al. as inventors, and entitled GUM COATINGS FOR CELL CULTURE, METHODS OF MANUFACTURE AND METHODS OF USE, which published patent application is hereby incorporated herein by reference for any and all purposes to the extent that it does not conflict with the present disclosure.

Coating of Polysaccharide Composition onto Substrate Surface

Polysaccharide layers, regardless of their composition, may be disposed on the surface of a cell culture article (base material or intermediate layer) in any suitable form. For example, the polysaccharide coatings may be in the form of a gel, hydrogel, film, powder, hydrofilm, or the like. The material may be opaque to transparent. Unprocessed and/or higher concentration gum material which contains impurities may form an opaque coating on a cell culture surface, while a more processed and/or lower concentration (i.e. centrifuged, filtered, or chemically purified) material may form a more transparent coating.

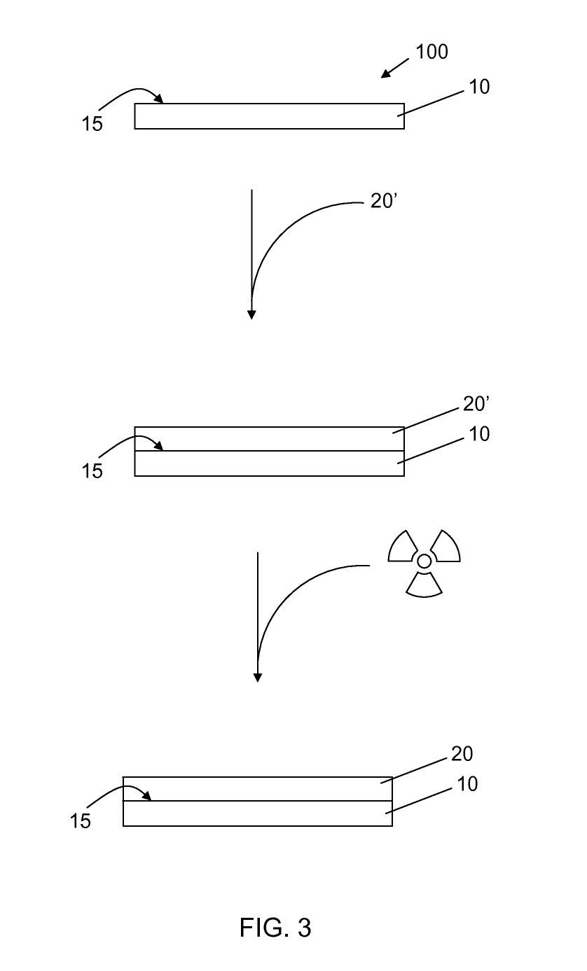

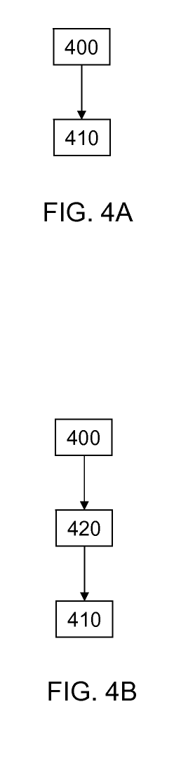

As shown in FIG. 3, after the polysaccharide 20′ is coated (for example in step 400, in FIG. 4A); i.e., generally disposed, on the surface 15 of the base material 10 the polysaccharide 20′ and article are irradiated (as in, for example, step 410 in FIG. 4A) with ionizing radiation to graft the polysaccharide 20′ to the surface 15 and form a cell culture surface 20.

General methods for grafting polysaccharide layers to a surface of a cell culture article are shown in FIGS. 4A-B. Generally, polysaccharide material is disposed on the surface of the article (400), which surface may be the surface of the base material or an intermediate layer, and the article and polysaccharide layer are exposed to ionizing radiation to graft the polysaccharide to the surface of the article (410). If the polysaccharide layer is disposed on the surface as a dry material, such as a powder or a film, the article and polysaccharide may be irradiated immediately.

If the polysaccharide layer is disposed on the surface as a solution, a wet hydrogel or hydrofilm, or the like, the polysaccharide may be dried (420) after disposing the galactomannan polymer on the surface of the cell culture article (400) and prior to exposing the article to ionizing radiation to graft the polysaccharide to the surface (410) (FIG. 4B). The concentration of the polysaccharide in solution before the application of the solution to a cell culture vessel may determine whether the material is a gel, hydrogel, powder, film or hydrofilm. A hydrogel or a hydrofilm may be a gel or film that has more water in the structure. In general, polysaccharide in solution or containing high amounts of water is dried until the water content is less than 60% of the weight of the polysaccharide composition. For example, the polysaccharide may be dried until the water content is less than 50%, less than 40%, less than 30% or less than 20%, or less than 10%, or less than 5% of the weight of the polysaccharide composition. Drying may occur under ambient conditions or with heating or vacuum, as desired.

As shown in the Examples below, galactomannan does not efficiently graft to the surface of the cell culture article when exposed to ionizing radiation in solution, but rather tends to depolymerize to small molecular weight oligomers or sugar monomers. Accordingly, it may be desirable for polysaccharides to be grafted to the surface in the solid state (e.g., less than 60%, than 50%, less than 40%, less than 30% or less than 20% water content).

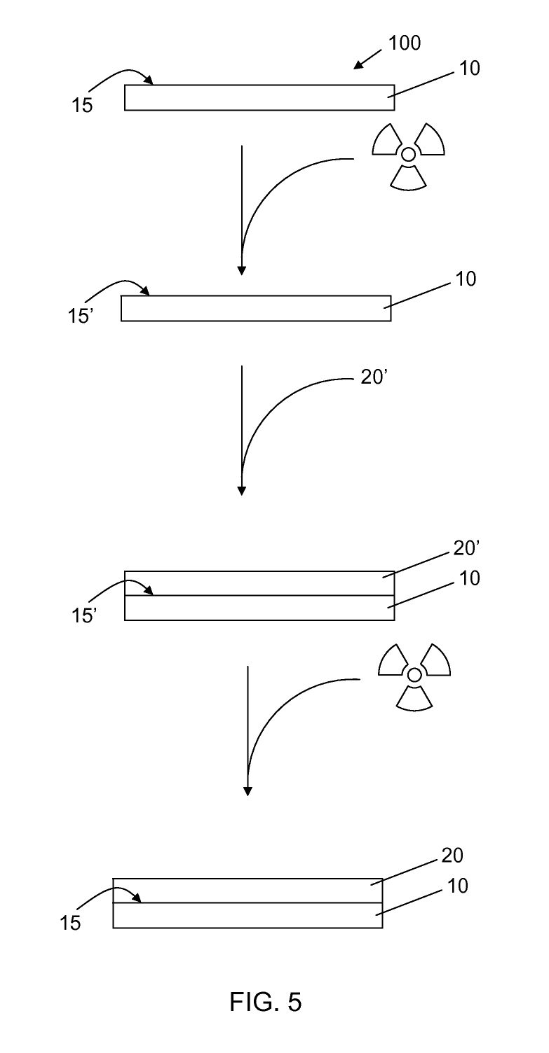

Referring now to FIG. 5, processes for grafting a polysaccharide layer to a surface of a cell culture article, where the surface of the article is pretreated with ionizing radiation, are shown. Without wishing to be bound by theory, it is believed that pretreatment of the surface 15 of a base material 10 of a cell culture article 10 results in a surface 15′ having free radicals to which a components of a polysaccharide composition 20′ may covalently bind. Upon subsequent radiation, it is believed that the polysaccharide 20′ cross-links forming a layer 20 that is less likely to solubilize and delaminate under cell culture conditions.

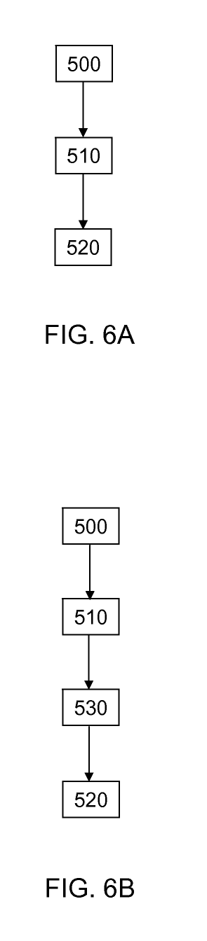

Overviews of embodiments of the general method depicted in FIG. 5 are shown in FIGS. 6A-B. Generally, the surface of a cell culture article is exposed to ionizing radiation (500) and the polysaccharide material is disposed on the irradiated surface of the article (510), which surface may be the surface of the base material or an intermediate layer, and the article and polysaccharide layer are exposed to further ionizing radiation (520), resulting in a cross-linked polysaccharide layer grafted to the surface of the article. If the polysaccharide layer is disposed on the surface as a dry material, such as a powder or a film, the article and polysaccharide may be irradiated immediately. If the polysaccharide layer is disposed on the surface as a solution, a wet hydrogel or hydrofilm, or the like, the polysaccharide may be dried 530 (see also 430, FIG. 4B), in some embodiments, prior to exposing the article to further ionizing radiation (520).

### Ionizing Radiation

Any suitable form of ionizing radiation that can cause free radical formation of polysaccharides and the surface of a cell culture article may be employed in accordance with the teachings presented herein. In various embodiments, e-beam radiation or gamma radiation (e.g., from ⁶⁰Co) is employed. In various embodiments, the article with coated polysaccharide is exposed to between about 1 kGy and 100 kGy of ionizing radiation, e.g. between about 5 kGy and about 50 kGy of radiation. In some embodiments, the article with coated polysaccharide is exposed to between about 10 kGy and about 40 kGy of radiation. In some embodiments, the article with coated polysaccharide is exposed to more than about 25 kGy of radiation.

Radiation doses for purposes of sterilizing high bioburden material may be in the range of 25 to 35 kGy. Of course, less ionizing radiation may be used to sterilize material that is not high bioburden. For example, a gamma radiation does or 10-20 kGy typically meets the bioburden requirement for cell culture substrates Thus, in some embodiments, the amount of ionizing radiation used to graft the polysaccharide layer to the surface of the culture article is sufficient to sterilize the article, eliminating an extra processing step.

Any suitable e-beam accelerator may be employed to graft polysaccharide layers to the surface of culture articles or sterilize the polysaccharide -grafted articles. Typically, e-beam accelerators are operated at between about 150 keV and 12 MeV. Some e-beam accelerators are capable of varying the energy at which they operate. In some embodiments, the cell culture articles are placed on a conveyer belt and moved through the e-beam accelerator. E-beam sterilization systems may contain sensors that allow for control of the speed of the conveyer if the e-beam current changes during processing so that the dose of e-beam radiation is held constant. Additionally, some e-beam systems are capable of holding the product at low temperatures to reduce the initiation of side chemical reactions.

Any suitable gamma irradiator may be employed to graft polysaccharide layers to the surface of culture articles or sterilize the polysaccharide-grafted articles. Often gamma irradiators emit gamma rays due to the decay of ⁶⁰Co. Because gamma rays are very penetrating and because gamma radiation tends to not break down packaging seals, the cell culture articles can be packaged prior to grafting and sterilization by gamma irradiation, reducing risk of subsequent contamination.

Of course other sources of ionizing radiation, such as X-rays, may be use for grafting or sterilization. For example, UV radiation may be used, in the presence of a photoinitiator, to graft the galactomannan to the surface. It is believed that UV radiation is not sufficient to efficiently graft galactomannan to the surface of a cell culture article in the absence of a photoinitiator.

Use of Coated Vessels

The cell culture articles produced according to the teachings presented herein can be used for culturing cells of any suitable type, such as primary cells, immortalized cells lines, groups of cells, tissues in culture, adherent cells, suspended cells, cells growing in groups such as embryoid bodies, eukaryotic cells, prokaryotic cells, or any other cell type. By way of example, articles produced according to the teachings presented herein can be used for culturing stem cells, committed stem cells, differentiated cells, and tumor cells. Examples of stem cells include, but are not limited to, embryonic stem cells, bone marrow stem cells and umbilical cord stem cells. Other examples of cells used in various embodiments include, but are not limited to, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells and cells of the immune system. Cell culture articles having a galactomannan coating may be particularly suitable for culturing hepatocytes (see, e.g., U.S. Patent Application Publication No. 2008/022526).

Cells cultured on articles produced in accordance with the teachings presented herein may be used to determine the interaction between the cells or cell lines and a factor or drug. The cells may be deposited on the polysaccharide layer. The deposited cells may be contacted with a factor, and the response produced by the deposited cells following contact with the factor may be identified.

With a known cell line immobilized on the coated substrates, it is possible to screen the activity of several drugs when the drug interacts with the immobilized cells. Depending upon the cells and drugs to be tested, the cell-drug interaction can be detected and measured using a variety of techniques. For example, the cell can metabolize the drug to produce metabolites that can be readily detected. Alternatively, the drug can induce the cells to produce proteins or other biomolecules. The substrates described herein provide an environment for the cells to more closely mimic the in vivo nature of the cells in an ex vivo environment.

Cell culture articles having a polysaccharide layer can be used in high throughput applications for analyzing drug/cell interactions. Cells can be grown on the coatings within multi-well plates used in high throughput applications. Cells can then be exposed to a drug, media can be removed from these high-throughput cultures, and the removed media can be analyzed for the presence of metabolites, proteins or other biomolecules. Increasing the population of cells per well, or increasing the in vivo-like nature of cells in culture, may serve to increase the value of signals measured by these techniques.

In various embodiments, hepatocyte cell cultures are cultured on a galactomannan layer articles and used as bio-artificial livers for use in compound toxicity evaluation, compound metabolisms, and protein synthesis. Hepatocytes have the ability to metabolize, detoxify, and inactivate exogenous compounds such as drugs and insecticides, and endogenous compounds such as steroids. The drainage of the intestinal venous blood into the liver requires efficient detoxification of miscellaneous absorbed substances to maintain homeostasis and protect the body against ingested toxins. One of the detoxifying functions of hepatocytes is to modify ammonia into urea for excretion.

In some embodiments, a method for culturing cells includes (a) depositing cells on a polysaccharide layer substrate described herein, and (b) culturing the cells on the substrate. It is contemplated that viable cells can be deposited on the coated substrates produced herein and cultured under conditions that promote tissue growth. Tissue grown (i.e., engineered) from any of the cells described above is contemplated with the coated substrates produced herein. The coated substrates can support many different kinds of precursor cells, and the substrates can guide the development of new tissue. The production of tissues has numerous applications in wound healing. It is contemplated that tissue growth can be performed in vivo or ex vivo.

In the following, non-limiting examples are presented, which describe various embodiments of grafting galactomannan layers to surfaces of cell culture articles.

### EXAMPLES

The following Examples describe processes wherein ionizing radiation; specifically gamma and e-beam irradiation, is used to immobilize galactomannan to a cell culture article substrate. The process may eliminate multiple post chemistry steps while achieving a robust cell culture surface and providing sterilization in one step. Gamma irradiated galactomannan (ex. locust bean gum, LBG) coated substrates are useful for hepatocyte cell culture, function, and cell based assays. In addition to providing the sterilized surface for cell attachment and growth, we also observe no delamination or dissolution of the coating and reduced or no loss (leaching) of the typical locust bean gum (LBG) polymer when testing the media extracted form coated substrates. This indicates that there is grafting of the polysaccharide (galactomannan) to the thermoplastic substrate (that is a source of free radicals and susceptible to free radical addition reactions) when exposed to gamma irradiation. We do observe a lower molecular weight component as an extractable within the first 3 days which could be a degradation product. During irradiation free radicals are generated that result in scission of polysaccharide chains forming chain breaks that are radicals which are quenched by radicals either (1) formed on the plastic surface resulting in grafting or (2) of another polymer chain resulting in interchain crosslinking. During this complex series of reactions, a majority of the polysaccharide (galactomannan) polymer is grafted (immobilized) to the plastic support while a very low % of the chain breaks that did not undergo grafting or crosslinking form low molecular weight compounds which could leach into solution as an extractable. These extractables are low molecular weight oligomeric sugars, sugars and/or sugar derivatives from polysaccharide repeat units (galactose, mannose) that are not harmful to liver cells, and may even be present as part of the composition of some cell culture medium.

### Example 1

### Grafting via Gamma Irradiation

Analysis was performed on locust bean gum (LBG) coated substrates, that were irradiated or not irradiated, to determine whether irradiation resulted (i) in grafting of galactomannan polymer to the substrates, thereby resulting in (a) less delamination or (b) less dissolution/extraction; (ii) significant degradation of the galactomannan polymer, as evidenced by a change in molecular weight; and (iii) any potential cytotoxic degradants.

Methods:

A solution of LBG (0.35-2% wt in pH 7.4 or deionized water) was pipetted 100-150 μl quantities onto a 96 well tissue culture treated polystyrene plate. The solution was dried at 90° C. for 1 h followed by 60° C. for 12 h followed by treatment with UV or gamma irradiation. UV (366 nm, 1 h) and gamma sterilized (10-18 KGy and 25-40 kGy) LBG coated plastic substrates were used to carry out a simulated cell culture experiments with the cell culture media only (without the cells) under typical cell culture conditions. Media was exchanged and collected on day 1 day 3 or 4 and then day 7 or 10. The collected media was used to run Gel Permeation Chromatography (GPC). GPC is a separation technique involving the transport of a liquid mobile phase through a column containing the separation medium, a porous material. GPC, also called size exclusion chromatography and gel filtration, affords a rapid method for the separation of oligomeric and polymeric species where the separation is based on differences in molecular size in solution. GPC is a good method for determining relative weight average molecular weight (Mw) and number average molecular weight (Mn) and polydispersity or distribution of the molecular weight (PDI). For graphical depicts of GPC analysis, the X axis depicts retention time (min) and the Y-axis depicts the refractive index of the solution (mV). Retention time is used to provide a relative molecular weight compared to standards run using the column. The peak area can be used to give the % quantity of material and the Y axis can be used to determine relative concentration.

In this Example, GPC is used to separate extract components, to quantify the % extractable (using peak area) and to determine whether the extractable is LBG (comparison of retention times). The resolution of LBG detection would depend on the GPC signal obtained for media.

GPC Experimental: The mobile phase was heated up to 85° C. and added to the samples. The samples then were stirred until they cooled down to room temperature. The samples were stored overnight at room temperature. The samples were run on a Waters Alliance 2695 System fitted with two Phenomenex TSK GMPWxL columns. The mobile phase consisted of 0.01 M NaCl and 200 ppm NaN3. The flow rate was 1 ml/min. The column temperature was maintained at 50° C. The detector was a Waters Refractive Index detector. The columns were calibrated against a series of pullulan polysaccharide standards (Polymer Labs). A linear calibration curve was obtained with an R²=0.999. The numbers printed on the chromatogram represent the molecular weight at the peak maximum (Mp).

Results:

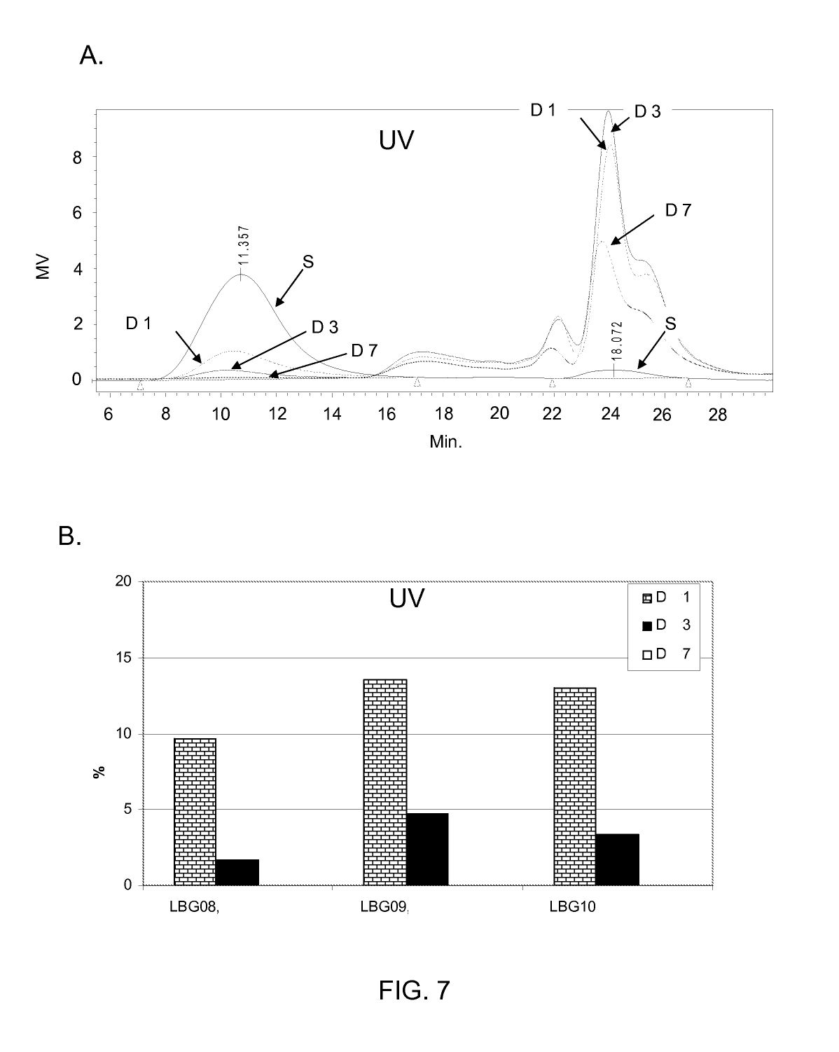

FIG. 7 shows results of GPC data depicting the amount of leaching of LBG into media from UW sterilized samples. In FIG. 7A, the X axis depicts retention time (min) and Y-axis depicts the refractive index of the solution (mV) for LBG standard and extractable containing media sample. LBG standard (S) appears within 9.5-13.5 min with a primary peak at 11.3-11.5 min retention time. Extractable containing media samples from day 1 (D1), day 3 (D3) and day 7 (D7) are shown. Extractable containing media sample collected at day 1 and 3 show a peak around 11.3 min which is comparable to LBG standard. Signal obtained for retention time>14 min is from media.

FIG. 7A shows that the LBG elutes as a broad peak with retention times in between 9.5-13.5 min due to the broadness of the band (PDI 1.4) the molecular weight could range from Mw of LBG 700-800 K. The X-axis depicts retention time (min), and the Y-axis depicts refractive index of the sample. In FIG. 7A, D1 indicates the elution profile at day 1; D3 indicates the elution profile at day 3; D7 indicates the elution profile at day 7; and S indicates the elution profile of a standard The LBG standard appears within 9.5-13.5 minutes with a primary peak at 11.3 to 11.5 min. Longer retention times depict lower Mw and lower retention times depict higher Mw. UV treated washed samples (LBG08, LBG09 and LBG10) show LBG as an extractable at day 1, day 3 but no LBG signal from the extraction at day 7 (see FIG. 7B, where the Y axis is the percent of peak area). The extractable amount is ˜14% and was quantified at 11.3 min retention time as with the control sample, as shown in Table 1.

TABLE 1
% Area for UV treated samples at retention time of 11.3 min (LBG peak)

| | % Area | | |
|---|---|---|---|
| Sample Number | Day 1 | Day 3 | Day 7 |
| LBG08 | 9.65 | 1.64 | 0 |
| LBG09 | 13.52 | 4.68 | 0 |
| LBG10 | 13.02 | 3.36 | 0 |

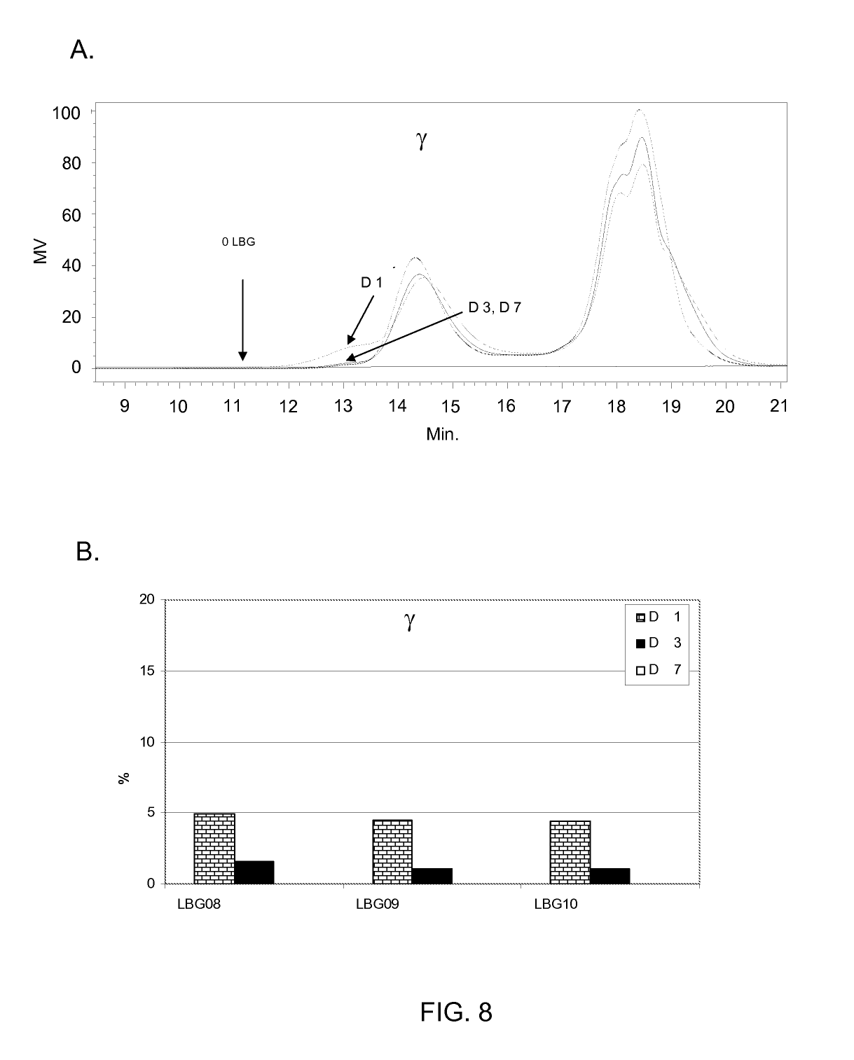

FIG. 8 shows results of GPC data depicting the amount of leaching of LBG into media from gamma (7) sterilized (25-40 kGy) samples. In FIG. 8A, The X-axis depicts retention time (min), and the Y-axis depicts refractive index of the sample. The LBG standard (not shown) appears within 9.5-13.5 minutes with a primary peak at 11.3 to 11.5 min. In FIG. 8A, D1 indicates the elution profile at day 1; D3 indicates the elution profile at day 3; and D7 indicates the elution profile at day 7. FIG. 8B is bar graph showing the percent of peak area of a GPC experiment for three gamma irradiated LBG samples at 13.5 minutes. As shown in FIG. 8A, gamma treated samples show no elution of LBG throughout the period but shows a peak at retention time of 13.5 min which could be a tailing band of low molecular weight oligomeric LBG, sugars and/or sugar derivatives resulting from chain breaks (or chain scission). This peak is observed only at day 1 and day 3. The amount eluted is <10% and were quantified at 13.5 min retention time (see FIG. 8B, where the Y axis is the percent of peak area, and Table 2). No extractable is seen at ˜11 min, indicating that there is grafting of LBG to the plastic under gamma irradiation conditions. The observation of an extractable ˜13 min indicates the formation of low molecular weight species of the polysaccharide (galactomannan) that elutes at higher retention times. Without being limited by theory, it may be that the low molecular weight species are formed via a complex set of reactions that occur as a result of the irradiation process. It is indirect evidence that the radical reactions that initiate grafting and crosslinking occurred.

TABLE 2
% Area for Gamma treated samples at retention time of 13.5 min

| | % Area | | |
|---|---|---|---|
| Sample Number | Day 1 | Day 3 | Day 7 |
| LBG08 | 4.94 | 1.59 | 0 |
| LBG09 | 4.51 | 1.05 | 0 |
| LBG10 | 4.42 | 1.08 | 0 |
| TCT | 0.37 | 0 | 0 |

TCT = corona treated polystyrene

Without being limited by theory, it might be that during irradiation, free radicals are generated that result in scission of polysaccharide chains forming chain breaks (radicals) which undergo addition reaction or are quenched by radicals either (1) formed on the plastic surface resulting in grafting or (2) of another polymer chain, resulting in interchain crosslinking. Based on the results presented herein, it appears that the irradiation process results in a majority of the polysaccharide (galactomannan) polymers being grafted (immobilized) to the plastic support, while a very low % of the chain breaks that did not undergo grafting or crosslinking form low molecular weight compounds. The species that result from the chain breaks leach into solution as an extractable. These extractables are believed to be low molecular weight oligomeric LBG or sugars, sugars and/or sugar derivatives from polysaccharide repeat units (galactose, mannose) that are not harmful to the cells. Primary hepatocytes and hepatocyte cell lines have been cultured on these surfaces up to day 14 and have remained viable as well as showed improved function compared to Collagen and comparable function compared to Matrigel™.

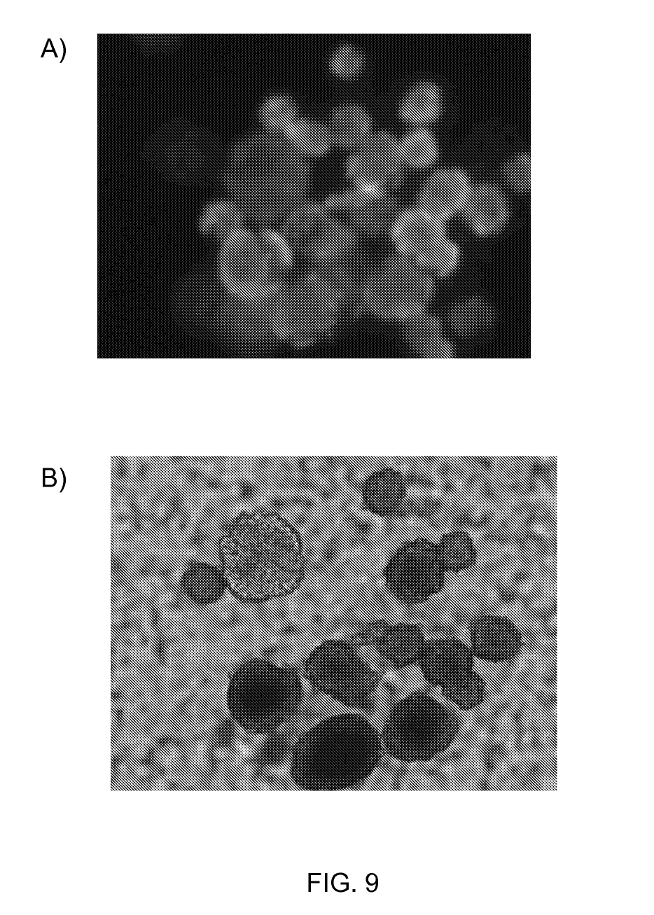

FIG. 9 shows images of cells cultured on gamma treated LGB surfaces at day 14. FIG. 9A is a fluorescent image at 50× of LIVE/DEAD® stained (Molecule Probes, Inc.) hepatocytes cells (HepG2-C3A cell line). The morphology is shown as clusters of cells or spheroids, and the white color spheroids indicate live cells. FIG. 9B is a brightfield image at 50× of the cells depicted in FIG. 9A, showing clusters of cells or spheroids. These cell images depicting spheroidal morphology, indicating cell viability, show that the extractables mentioned above are not harmful to the cells and that cells remain viable during the 14 days of culture.

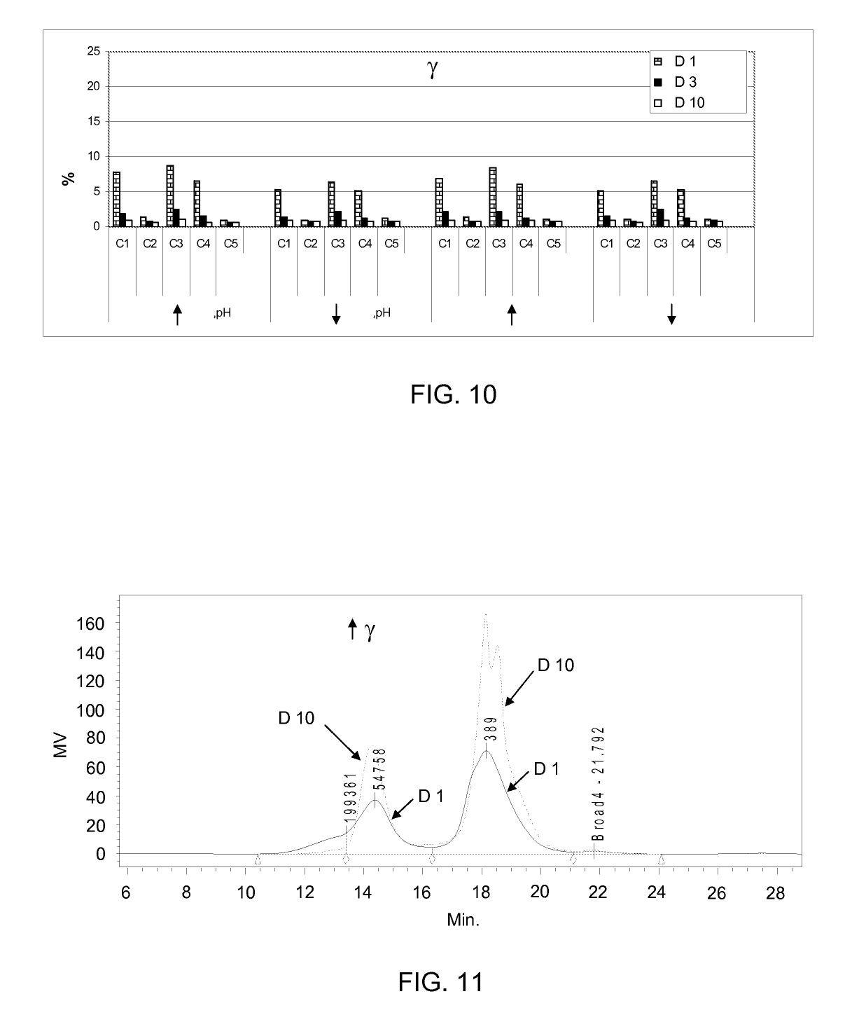

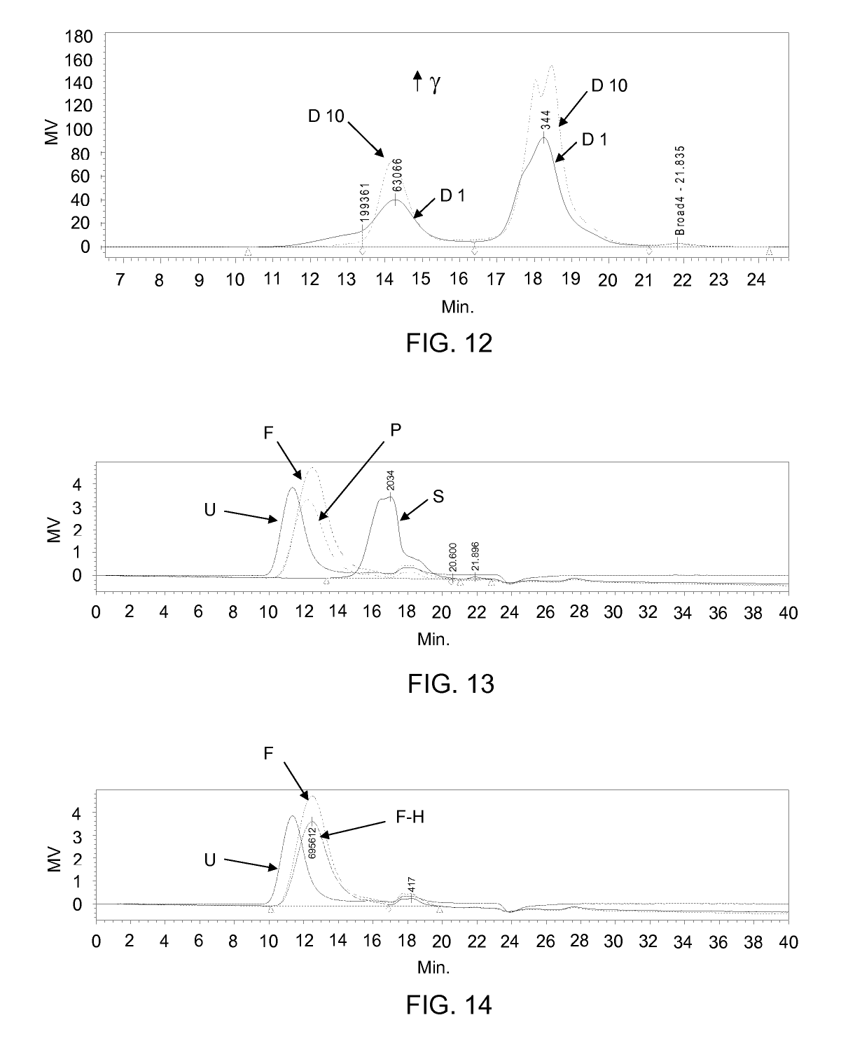

FIGS. 10-12 show low ↓ (10-18 kGy) and high gamma sterilized ↑ (25-40 kGy) LBG coating data. FIG. 10 is a bar graph showing results of GPC experiments LBG coated articles subjected to low (10-18 kGy) and high (25-40 kGy) dose gamma radiation LBG coated articles at a retention time of 13.5 minutes. The results shown compare pH 7.4 and deionized water-based LBG coatings. In FIG. 10, T indicates high dose gamma radiation; ↓ indicates low dose gamma radiation; using both pH 7.4 and deionized water based LBG coatings. C1 indicates 100 μl solution coated and dried at 90° C. for 1 h followed by 60° C. for 12 h; C2 indicates 100 μl added where 50 μl was aspirated after 15 minutes and dried at 90° C. for 1 h followed by 60° C. for 12 h; C3 indicates 100 μl coated then dried 60° C. and recoated with 100 μl and dried at 90° C. for 1 h followed by 60° C. for 12 h of solution; C4 indicated indicates 100 μl solution coated and dried at 60° C. for 12 h; and C5 indicates tissue culture treated plate (no coating). C1-C5 for both high and low gamma treatment (solution made from pH 7.4 or deionized water) were incubated with media 10 days while aspirating the media on day 1, 3 and day 10 and replacing with fresh media. The brick filled bars in FIG. 10 represent the percent area on day 1; the black bars represent % area on day 3; and the white bars represent % area on day 10. FIGS. 11-12 are representative examples of graphs of GPC sample data that was collected and used in the calculations for the results presented in FIG. 10. FIG. 11 is a representative GPC sample of a pH 7.4 LBG coated article subjected to high dose gamma radiation. FIG. 12 is a representative GPC sample of a deionized water-based LBG coated article subjected to high dose gamma radiation. In FIGS. 11-12, D1 indicated the elution profile at day 1; and D10 indicates the elution profile at day 10. In this experiment surfaces of different thickness, heat treatment and coatings made from pH 7.4 and deionized water were subjected to two different gamma doses to see the effect of gamma irradiation on different coatings.

For the studies that produced the results presented in FIGS. 7-12, coatings were made using LBG solutions prepared using pH 7.4 and deionized water coating solutions. A solution of LBG (0.35-2% by weight in pH 7.4 or deionized water) was pipetted 100-200 μl quantities onto a 96 well tissue culture treated polystyrene plate. The solution was dried at 90° C. for 1 h followed by 60° C. for 12 h (or in some instances dried only at by 60° C. for 12 h ) followed by treatment with UV or gamma irradiation. UV (366 nm, 1 h) and gamma sterilized (10-18 KGy and 25-40 kGy) LBG coated plastic substrates. Similar to results presented in FIG. 8, which depicts coatings treated with high gamma dose of 25-40 kGy, FIG. 10 shows that both low and high gamma-treated samples show no elution of LBG throughout the period. However, as shown in FIGS. 11-12, a peak at retention time of 13.5 min was observed, which could be a tailing band of low molecular weight oligomeric LBG, sugars and/or sugar derivatives resulting from chain breaks (or chain scission). This peak is observed only at day 1 and day 3. FIG. 10 further shows the % extractable is <10% for all the conditions and were quantified at 13.5 min retention time. Since no extractable is seen at ˜11 min, the results indicate that there is grafting of LBG to the plastic under gamma irradiation conditions. As shown in the results presented in FIG. 10, effective grafting with a low percentage of extractables was observed for all of the test conditions, which results are exemplified in FIGS. 11-12 which show similar elution profiles for pH 7.4 and deionized water-based LBG coatings.

In order to understand the low molecular band observed as an extractable for LBG coated plastics, an experiment was carried out using LBG powder, LBG free standing film (no plastic support) and LBG solution in glass vials. These were subjected to gamma irradiation or heat treatment followed by gamma irradiation to simulate typical process conditions for the LBG coated polystyrene vessels described above. In all cases radicals can be formed by irradiation, however the main difference between these samples and the LBG coated plastic vessel is the absence of the plastic support that is a source of free radicals and susceptible to free radical addition reactions.

Table 3 summarizes GPC results for LBG samples with no treatment, LBG powder and free-standing films treated with gamma irradiation and heat treated followed by gamma irradiation. In general, gamma treatment and the combination of heat (95° C., 1 h then 60° C.) followed by gamma treatment of LBG in solid state (powder and film) yield samples that showed decrease in Mw and Mn (retention time at 12.2-12.5 min, changed from 11.3 min) and an increase in the molecular weight distribution (PDI) (see FIGS. 13-14). FIGS. 13-14 are representative graphs of GPC sample data that was collected and used for calculations of the results presented in Table 3. In FIGS. 13-14, F indicates a solution made from free standing LBG films that were gamma irradiated; P indicates solution made from LBG powders that were gamma irradiated; S indicates solution made from LBG in solution (7.5 mg/ml in deionized water) that were gamma irradiated; U indicates solution made from untreated (no gamma radiation) LBG powder; and F-H indicates solution made from a free standing LBG films that were heated and gamma irradiated. The decrease in molecular weight, first peak for both Mn and Mw in Table 3 with respect to LBG (reference) supports chain scission as radicals are generated. It is worth noting that the polydispersity (PDI) of the first peak increased significantly (from 1.4 to 3.8-6.2) after gamma and heat/gamma treatment describing a broader distribution of LBG chains of varying sizes (Table 3). The larger molecular weight chains in these samples (P and F, Mn:126-65K) eluted at retention time 12.2-12.5 min (FIGS. 13 and 14) are not observed in the extractions from the gamma irradiated LBG coated plastic vessels, however the tail of this peak overlaps with the shoulder (small broad peak) observed in the chromatograph (FIG. 8) for the LBG coated plastic vessels at retention time 13.5 min representing low molecular weight oligomeric sugars and sugar derivatives as a very low percentage of the extracts of those samples. Without being limited by theory this may suggest that there is a specific role of the plastic substrate, indicating that the plastic substrate participates in the radical addition and quenching reactions, and that there is immobilization of larger molecular weight LBG to the plastic substrate.

TABLE 3
GPC Results

| | Retention | | | | |
|---|---|---|---|---|---|
| Sample | time | Mn | Mw | PDI | % Area |
| LBG1 | 11.3 | 536,900 | 764,900 | 1.4 | 93.54 |
| (reference) | 18.2 | 500 | 620 | 1.2 | 6.46 |
| LBG powder | 12.2 | 126,900 | 466,200 | 3.8 | 95.31 |
| gamma | 18.2 | 460 | 580 | 1.3 | 4.69 |
| treated (P) | | | | | |
| LBG powder | 12.5 | 78,800 | 408,000 | 5.2 | 94.78 |
| heated then | 18.2 | 490 | 640 | 1.3 | 5.22 |
| gamma | | | | | |
| treated | | | | | |
| LBG film | 12.5 | 65,900 | 405,800 | 6.2 | 92.26 |
| gamma | 17.8 | 510 | 690 | 1.4 | 7.74 |
| treated (F) | | | | | |
| LBG film | 12.5 | 79,300 | 409,400 | 5.2 | 94.11 |
| heat treated | 18.2 | 500 | 690 | 1.4 | 5.89 |
| then gamma | | | | | |
| (F-H) | | | | | |
| LBG solution | 17.0 | 1500 | 4400 | 2.9 | 95.69 |

| | | | |
|---|---|---|---|
| gamma | 21.9 | Not quantified | 4.31 |
| treated (S) | | | |

The differences in GPC data for solutions made from gamma irradiated LBG (powder and free-standing film) and gamma irradiated LBG solution is significant. Table 3 shows that LBG solution heat treated and then gamma treated showed a very sharp decrease in the molecular weight, Mw from 765K to 4K (retention time of 17 min, changed from 11.3) suggesting the irradiation caused degradation to sugar molecules and derivatives. There is no evidence of polymeric LBG or crosslinked LBG which would appear as insoluble gels or precipitate in the sample. The apparent complete conversion of LBG polymer to small sugar molecules and/or derivatives on exposure to irradiation in low concentration solution state is further supported by the PDI which is lower than in the cases where LBG was gamma treated in powder or film state. This indicates that solid state (e.g., powder or film) of LBG is advantageous to achieve immobilization and crosslinking as the desired outcome of the competing events (radical addition reaction, chain scission and radical termination) during gamma irradiation of LBG on a plastic substrate that participates in free radical reactions. Of course, based on these results, it may be expected that other substrates that can participate in the free radical reactions could be advantageously used.

### Example 2

### Grafting via E-Beam Irradiation

The ability of electron beam (e-beam) radiation to graft locust bean gum to a plastic substrate was studied. The effects of pretreatment of the cell culture plate with e-beam irradiation (prior to coating LBG) and post-treatment (after coating with LBG) were evaluated. It is believed that pre-treatment may cause free-radicals to form on the surface of the cell culture plate, allowing the locust bean gum to graft to covalently bind to the surface, resulting in reduced delamination. It is also believed that post treatment may cause the locust bean gum to cross-link, resulting in reduced delamination due to decreased solubility. These hypotheses were tested in this example.

Briefly, a set of 18 untreated polystyrene dishes were coated with a 0.75% (w/v) solution of LBG. The parts were irradiated per the matrix shown in Table 4.

TABLE 4
E-beam exposure conditions for Polystyrene 96 well plate

Ebeam Pretreatment

| | | 0 KGy | 5 KGy | 40 KGy |
|---|---|---|---|---|
| Ebeam Post | 0 KGy | 3 plates | 3 plates | 3 plates |
| Treatment | 5 KGy | 3 plates | 3 plates | 3 plates |
| | 40 KGy | 3 plates | 3 plates | 3 plates |

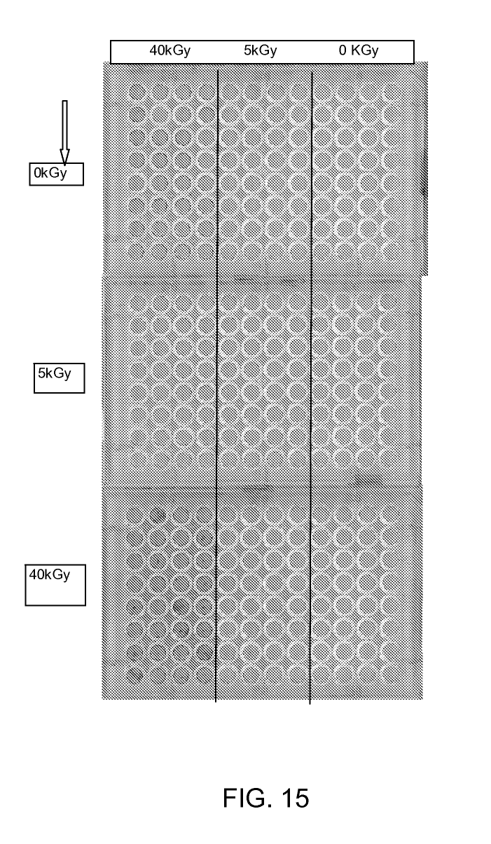

The treated parts were soaked in a buffer solution for 4 days at 37° C. to simulate cell culture. After the soaking period, they were stained with crystal violet dye and imaged photographically, as illustrated in FIG. 15. The left column of wells separated by the dark vertical line in FIG. 15 were pretreated with 40 kGy gamma radiation; the middle column was pretreated with 5 kGy gamma radiation; and the left column was not pretreated with gamma radiation, as shown. The top row of wells identified by the dark vertical line in FIG. 15 was subjected to no gamma radiation after LBG coating; the middle row was subjected to 5 kGy gamma radiation after LBG coating; and the bottom row was subjected to 40 kGy gamma radiation after LBG coating, as shown. The images were visually evaluated on a scale of 0 to 5 for delamination, with 0 being the highest delamination and 5 being the least delamination. The results are summarized in Table 5.

TABLE 5
Delamination of LBG coating from polystyrene as a function
of pretreatment and post-treatment with e-beam irradiation

| | | Pretreatment | | |
|---|---|---|---|---|
| | | No Ebeam | 5 KGy dose | 40 KGy dose |
| Post treatment | No Ebeam | 0 | 0 | 1 |
| | 5 KGy dose | 1 | 1 | 1 |
| | 40 KGy dose | 1 | 2 | 3 |

Pretreatment with a dose of 40 KGy and post-treatment with a dose of 40 KGy resulted in the highest adhesion of the LGB to the polystyrene surface, indicating that e-beam irradiation is a feasible process for cross-linking and grafting galactomannans to a plastic surface.

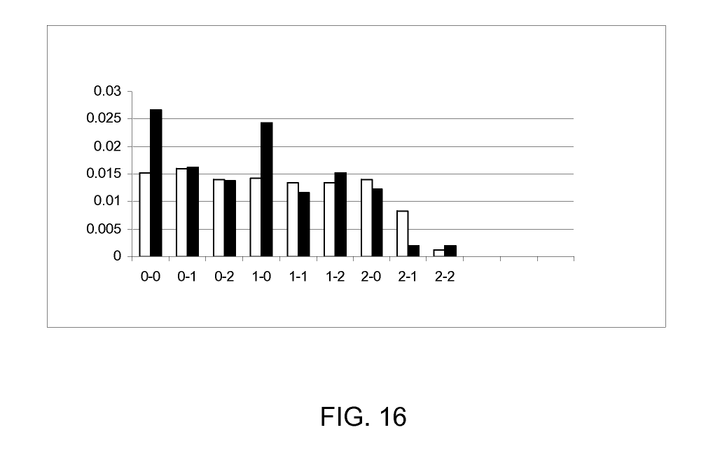

In an effort to quantify the delamination/material removed as a result of exposure to e-beam, locust bean gum coated plates were irradiated using the same scheme listed in Table 4 above. Phosphate buffered saline was added to each well in every plate and incubated at 37° C. for 3 days. The supernatant solution was pipetted out, and solution from all wells of a given condition in each plate was combined and dried. The remaining locust bean gum residue was weighed to see the quantity of locust bean removed from or leaving the bottom of each well. This weight is an indication of the degree of lamination of the LBG to the bottom of the well. That is, a higher quantity of LBG removed from the bottom of each well indicates less effective grafting of the material to the bottom of the well. A lower quantity of LBG removed from the bottom of the well indicates more effective grafting of the material to the bottom of the well as a result of Ebeam irradiation. The lower the weight of locust bean, the higher the degree of grafting. The results are shown in FIG. 16. The graph legend in FIG. 16 is as follows: 0-0 is 0 KGy pretreatment and 0 KGy post treatment; 0-1 is 0 KGy pretreatment and 5 KGy post treatment; 0-2 is 0 KGy pretreatment and 40 KGy post treatment; 1-0 is 5KGy pretreatment and 0 KGy post treatment; 1-1 is 5 KGy pretreatment and 5 KGy post treatment; 1-2 is 5 KGy pretreatment and 40 KGy post treatment; 2-0 is 40KGy pretreatment and 0 KGy post treatment; 2-1 is 40 KGy pretreatment and 5 KGy post treatment; 2-2 is 40 KGy pretreatment and 40 KGy post treatment. FIG. 16 Y-axis show the weight (in grams) of LBG dissolved in PBS after a 3 days of soaking at 37° C. after various pretreatment and post-treatment conditions of e-beam irradiation. As shown in FIG. 16, 96 well plates coated with LBG when pre-irradiated at a dose of 40 KGy and post irradiated at 5 KGy or 40 KGy only lose 0.0055 g and 0.0015 g of locust bean gum respectively.

The Examples presented herein describe the use of ionizing radiation to graft solid state polysaccharides to cell culture substrates, immobilizing the polysaccharides to the cell culture article, and improving the integrity of the polysaccharide coating under cell culture conditions. One advantage is that the immobilization of the polysaccharide to the cell culture substrate can be done in a typical sterilization step.

Thus, embodiments of IRRADIATION INDUCED GRAFTING OF POLYSACCHARIDES TO CELL CULTURE VESSELS are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method for grafting a polysaccharide to a surface of a cell culture article, comprising:
contacting the surface of the article with the polysaccharide in a dry form; and
exposing the article and contacted dry polysaccharide to ionizing radiation to graft the polysaccharide to the surface of the article.

2. The method of claim 1, wherein the dry polysaccharide is in the form of a powder.

3. The method of claim 1, wherein the dry polysaccharide is in the form of a film.

4. The method of claim 1, further comprising contacting the surface of the article with the polysaccharide in a solvent and removing the solvent to result in the contacting the surface of the article with the polysaccharide polymer in a dry form.

5. The method of claim 4, wherein the polysaccharide is dried such that the weight of the solvent is less than 50% of the weight of the polysaccharide.

6. The method of claim 1, wherein the polysaccharide comprises a galactomannam polymer.

7. The method of claim 6, wherein the galactomannan polymer comprises a ratio of mannose to galactose of about 1:1 to about 5:1.

8. The method of claim 6, wherein the galactomannan polymer comprises a ratio of mannose to galactose of about 4:1.

9. The method of claim 6, wherein the galactomannan polymer comprises locust bean gum.

10. The method of claim 6, wherein the galactomannan polymer consists essentially of locust bean gum.

11. The method of claim 1, wherein the surface of the cell culture article is formed from a thermoplastic material.

12. The method of claim 1, wherein the surface of the cell culture article is formed from a polystyrene polymer, polypropylene polymer or a cyclic polyolefin polymer.

13. The method of claim 1, wherein the surface of the cell culture article is plasma or corona treated.

14. The method of claim 1, wherein the polysaccharide consists essentially of locust bean gum, and wherein the surface of the cell culture article is formed from a polystyrene polymer, polypropylene polymer or a cyclic polyolefin polymer.

15. The method of claim 1, wherein exposing the article and contacted dry polysaccharide comprises exposing the article and the polysaccharide to a dose of gamma radiation of between about 5 kGy and about 50 kGy of radiation.

16. The method of claim 1, wherein exposing the article and contacted dry polysaccharide comprises exposing the article and the polysaccharide to a dose of electron beam radiation of between about 1 kGy and about 100 kGy of radiation.

17. The method of claim 1, wherein exposing the article and contacted dry polysaccharide comprises exposing the article and the polysaccharide to between about 10 and about 18 kGy of ionizing radiation.

18. The method of claim 1, wherein exposing the article and contacted dry polysaccharide comprises exposing the article and the polysaccharide to between about 25 and about 40 kGy of ionizing radiation.

19. The method of claim 1, wherein exposing the article and contacted dry polysaccharide comprises exposing the article and the polysaccharide to a sufficient amount of ionizing radiation to sterilize the article and the polysaccharide.

20. The method of claim 1, further comprising packaging the article for cell culture use without extracting extractables from the polysaccharide and surface of the article following exposing to radiation.

21. The method of claim 1, further comprising exposing the surface of the article to ionizing radiation prior to contacting the surface with the polysaccharide.

22. A cell culture article made according to the method of claim 1.

23. A cell culture article comprising:
a substrate surface; and
a galactomannan polymer covalently bonded to the surface.

24. The article of claim 23, wherein the article supports the culture of liver cells.
